(19) United States
(12) Patent Application Publication
(10) Pub. No.: US 2020/0016094 A1
(43) Pub. Date: Jan. 16, 2020

(54) THERAPEUTIC CANNABINOID DERIVATIVES COMPOSITION AS HISTAMINE 2 (H2) BLOCKING AGENTS

(71) Applicants: Henry Lowe, Kingston (JM); Ngeh J. Toyang, Columbia, MD (US)

(72) Inventors: Henry Lowe, Kingston (JM); Ngeh J. Toyang, Columbia, MD (US)

(21) Appl. No.: 16/512,340

(22) Filed: Jul. 15, 2019

Related U.S. Application Data
(60) Provisional application No. 62/698,716, filed on Jul. 16, 2018.

(51) Int. Cl. A61K 31/05

(52) U.S. Cl. CPC A61K 31/05

(57) ABSTRACT

A cannabinoid-based pharmaceutical composition and therapy for the prevention and/or treatment of a gastrointestinal tract condition, including gastritis, acid reflux, gastroesophageal reflux disease, heartburn, and associated complications. The composition may include a natural or synthetic cannabidiol molecule.

### CROSS-REFERENCE TO RELATED APPLICATION(S)

The present application derives priority from U.S. provisional application Ser. No. 62/698,716 filed 16 Jul. 2018.

### BACKGROUND OF THE INVENTION

### Field of the Invention

The present invention relates to cannabinoid derivatives and, more particularly, to cannabis cannabinoid derivatives or the pharmaceutically acceptable salt thereof, which may be used in a pharmaceutical composition for preventing and treating conditions of the gastrointestinal tract (GIT), including gastroesophageal reflux disease (GERD) and associated complications.

### 2. DESCRIPTION OF THE BACKGROUND

GERD is a long-term condition where stomach contents come back up into the esophagus resulting in symptoms or complications. Symptoms include the taste of acid in the back of the mouth, heartburn, bad breath, chest pain, vomiting, breathing problems, and wearing of teeth. Although GERD is primarily a motor disorder, the injurious effects of gastric acid are central to the pathogenic process of esophagitis, and the severity of disease correlates with the degree and duration of esophageal acid exposure. In the majority of patients with mild disease, esophageal acid exposure occurs predominantly during post-prandial periods.

In the Western world, between 10 and 20% of the population are affected by GERD Hershcovici, T. and Fass, R., Pharmacological management of GERD: where does it stand now?, Trends in Pharmacological Sciences 32(4) (April 2011), pp. 258-264. The condition was first described in 1935 by the American gastroenterologist Asher Winkelstein following the classic earlier description of the symptoms in 1925 by Julius Friedenwald and Maurice Feldman. Arcangelo, Virginia Poole and Peterson, Andrew M., Pharmacotherapeutics for Advanced Practice: A Practical Approach, Lippincott Williams & Wilkins (2006), p. 372.; Granderath, Frank Alexander, et al., Gastroesophageal Reflux Disease: Principles of Disease, Diagnosis, and Treatment, Springer Science & Business Media (2006), p. 161.

Treatment is typically via lifestyle changes, medications, and sometimes surgery. Lifestyle changes include not lying down for three hours after eating, losing weight, avoiding certain foods, and stopping smoking. Medications include antacids, H2 receptor blockers, proton pump inhibitors (PPIs), and prokinetics. Surgery may be an option in those who do not improve with other measures.

Since their discovery in the late 1980s, PPIs have been largely used for the treatment of acid related disorders. The efficacy of PPIs is superior to H2 receptor antagonists (H2RA) for severe GERD complications. Bamberg, P., et al., A meta-analysis comparing the efficacy of omeprazole with H2-receptor antagonists for acute treatment of duodenal ulcer in Asian patients, J. Gastroenterol. Hepatol., 7 (1992), pp. 577-585; Gisbert., J. P, et al., Proton pump inhibitors versus H2-antagonists: a meta-analysis of their efficacy in treating bleeding peptic ulcer Aliment, Pharmacol. Ther., 15 (2001), pp. 917-926. The use of PPIs is recommended in current guidelines for the treatment of GERD. DeVault, K. R. and Castell, D. O., Updated guidelines for the diagnosis and treatment of gastroesophageal reflux disease, Am. J. Gastroenterol., 100 (2005), pp. 190-200.

PPIs are also recommended for peptic ulcer prevention in patients receiving NSAIDs and eradication protocols. Malfertheiner, et. Al., Current concepts in the management of Helicobacter pylori infection: the Maastricht III Consensus Report Gut, 56 (2007), pp. 772-781. Some guidelines recommend treating symptoms with an H2 antagonist before using a PPI because of cost and safety concerns. Consumer Reports, Drugs to Treat Heartburn and Stomach Acid Reflux: The Proton Pump Inhibitors—Comparing Effectiveness, Safety, and Price, Drug Effectiveness Review Project (May 2010).

Studies have found that PPIs reduce stomach acid more over time than antacids or H2 blockers, but PPIs are not intended for the immediate relief of heartburn. Antacids and H2 blockers actually do a better job at that. The main advantage of treatment with PPIs, in addition to reducing stomach acid for longer periods of time, is that they treat people diagnosed with GERD or ulcers. H2 blockers indirectly suppress gastric acid and pepsin by binding to the H2 receptors and interfering with their stimulation of basal, nocturnal, and pentagastrin secretion, including insulin and meal induced acid secretion.

The recent surge in interest in medical cannabis has led to interest in evaluating and validating the therapeutic potential of cannabis and its metabolites against various diseases. Cannabinoids and terpenes are found in cannabis plants. Cannabinoids are known for their antioxidant and anti-inflammatory health benefits and have relevant pharmacological activities such as antidiabetic, antiallergic, antibiotic, and antidiarrheal activities. Further, they exhibit beneficial pharmacological properties against central nervous system (CNS) disease and cancer. Cannabinoids such as Tetrahydrocannabinol (THC) and Cannabidiol (CBD), and terpenes like myrcene and limonene, also produce a range of effects. Unfortunately, much attention on Cannabis is focused on its recreational use as a psychoactive drug. However, CBD is a nonpsychoactive cannabinoid credited for several pharmacological properties. It is known to have beneficial effects against inflammation/pain, neurological conditions, cancer, and other ailments. Campos, A. C., et al., Cannabidiol, Neuroprotection and Neuropsychiatric Disorders, Pharmacol. Res., 112 (2016), pp. 119-127; Fernandez-Ruiz, J., et al., Cannabidiol For Neurodegenerative Disorders: Important New Clinical Applications for This Phytocannabinoid?, Br. J. Clin. Pharmacol., 75 (2013), pp. 323-333; Mechoulam, R., et al., Cannabidiol—Recent Advances, Chem. Biodivers, 4 (2007), pp. 1678-1692; McPartland, J. M. and Russo, E. B., Cannabis and Cannabis Extracts: Greater Than The Sum Of Their Parts?, J. Cannabis. Ther. 1 (2012), pp. 103-132.

Other researchers have studied the effect of other synthetic cannabinoids on GIT complications. Appendino and colleagues synthesized a series of quinone cannabinoids and reported their modulatory activity on mammalian Perixome Proliferator Activated Receptors (PPARs). Appendino, Giovanni, et al., Cannabidiol quinone derivatives, U.S. Pat. No. 9,701,618. PPARs control the expression of networks of genes involved in adipogenesis, lipid metabolism, inflammation, and maintenance of metabolic homeostasis. In various studies, activators of a particular PPAR subtype, PPARγ, show protection against gastric ulcers and also accelerated the ulcer healing in gastric ulcers in rats. Both PPARγ and another PPAR subtype, PPARα, may be a target for gastric ulcer therapy. Saha, Lekha, Role of peroxisome proliferator-activated receptors alpha and gamma in gastric ulcer: An overview of experimental evidences, World J. Gastrointest. Pharmacol. Ther. 6(4) (Nov. 6, 2015), pp. 120-126. Gastric ulcers and GERDs are both common acid-related disorders. While gastric ulcers or gastritis is mainly a stomach disease, GERD is a disease of the esophagus. Gastritis is one of the several diseases that Appendino reported could be managed by the use of synthetic quinone cannabinoids that are modulators of PPARs.

### SUMMARY OF THE INVENTION

It is, therefore, an object of the invention to provide a pharmaceutical composition that is a natural or synthetic CBD molecule for the prevention and/or treatment of GIT conditions, which may include gastritis, acid reflux, GERD, heartburn, and associated complications.

In accordance with the foregoing objects, embodiments of the present invention provide a cannabinoid-based pharmaceutical composition for the prevention and/or treatment of GIT conditions, having the following structure or a pharmaceutically acceptable salt thereof:

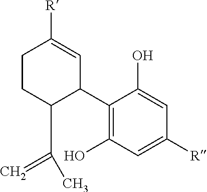

In the above composition, R′ may be CH₃ and R″ may include various substituents. For example, R″ can be: (1) an alkyl of 5 to 12 carbon atoms; (2) a group —O—R′″, wherein R′″ is an alkyl of 5 to 9 carbon atoms; (3) a group —O—R′″, wherein R′″ is an alkyl having a phenyl group at a terminal atom; or (4) a group —(CH₂)ₙ—O-alkyl, wherein n is an integer from 1 to 7 and the alkyl has 1 to 5 carbon atoms.

In another embodiment, a cannabinoid-based pharmaceutical composition for the prevention and/or treatment of GIT conditions, having the following structure, or a pharmaceutically acceptable salt thereof:

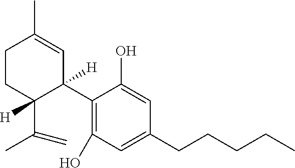 [SMILES: C=C(C)[C@@H]1CCC(C)=C[C@H]1c1c(O)cc(CCCCC)cc1O]

A therapy for the prevention and/or treatment of GIT conditions using a cannabinoid-based pharmaceutical composition shown above can include administering either or both of the above compositions to a patient.

### BRIEF DESCRIPTION OF THE DRAWINGS

Other objects, features, and advantages of the present invention will become more apparent from the following detailed description of the preferred embodiments and certain modifications thereof when taken together with the accompanying drawings in which:

FIG. 1 is a structural formula for an example cannabinoid-based pharmaceutical composition for the prevention and treatment of GIT conditions, according to embodiments of this invention.

FIG. 2 is a structural formula for an example cannabinoid-based pharmaceutical composition for the prevention and treatment of GIT conditions, according to embodiments of this invention.

FIG. 3 is a flow diagram for a method for the prevention and/or treatment of GIT conditions using a cannabinoid-based pharmaceutical composition, according to embodiments of this invention.

FIG. 4 is a table showing an example of antagonist activity of CBD against H2, according to embodiments of this invention.

### DETAILED DESCRIPTION OF THE PREFERRED EMBODIMENTS

Reference will now be made in detail to preferred embodiment of the present invention, examples of which are illustrated in the accompanying drawing.

Embodiments of present invention include a group of cannabinoid-based pharmaceutical compositions for the prevention and/or treatment of GIT conditions, which may include gastritis, acid reflux, GERD, heartburn, and associated complications.

Most of the studies on CBD and cannabis, in general, have focused on the neuroprotective as well as anti-inflammatory properties. The studies have focused on the effect of natural and synthetic cannabinoids on the cannabinoid receptors CB1 and CB2. Of the two receptors, only CB1 has been detected on the enteric nerves in the GIT, and their pharmacological properties include gastroprotection through gastrointestinal motility and secretion reductions. Di Carlo, Giulia and Izzo, Angelo, Cannabinoids for gastrointestinal diseases: potential therapeutic applications, Expert Opinion on Experimental Drugs., 12(1) (2003).

Unlike CB1 receptors, CB2 receptors in the GIT are mostly present in immunocytes. CB2 receptors are not involved in the physiological regulation of the motility of the GIT but are involved in inflammatory processes. Schicho, Rudolf and Storr, Martin, Alternative targets within the endocannabinoid system for future treatment of gastrointestinal diseases, Canadian Journal of Gastroenterology, 25(7) (2011). The human body and the GIT, in particular, also produce endogenous cannabinoids, including anandamide and 2-aracidonylglycerol. The pharmacological modulation of these cannabinoids may be also therapeutically useful in the management of GIT conditions.

Unfortunately, the physiological regulation of the endocannabinoids system to manage GIT complications using medications is yet to be achieved. Based on existing scientific evidence, of the two major cannabinoids, only THC can be seen to have potential via an agonist effect on the CB1 receptor. CBD has no effects on the CB1 receptor and is known to be an inverse agonist of the CB2 receptor. Despite the possible benefits of CB1 modulation in managing complications of the GIT, CB1 is yet to be considered a therapeutic target for GERD. Unlike THC, which has been shown to have antagonist effect against H1, CBD has a potent antagonist effect against H2. As already described above, H2 blockers and PPIs are the only two major therapeutic targets for GERD. Current literature has no reports on the blockage of H2 by CBD, making the discovery of this hitherto unknown property of CBD a novelty.

The present inventors have established the therapeutic properties of CBD and have proven the efficacy of CBD against H2, a validated target for the treatment of GERD, using various pharmacologic assays.

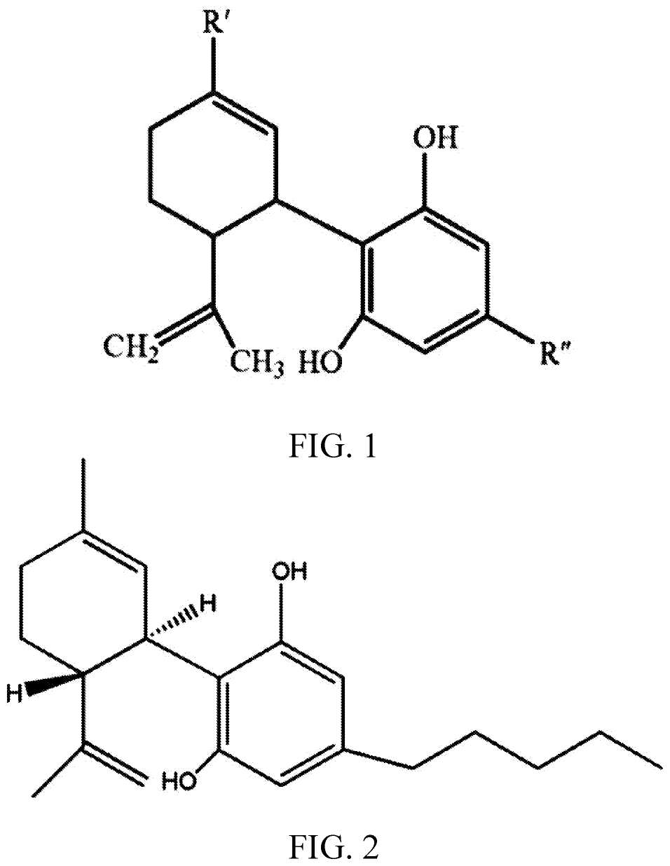

FIG. 1 is a structural formula for an example cannabinoid-based pharmaceutical composition for the prevention and/or treatment of GIT conditions, according to embodiments of this invention. In the composition shown in FIG. 1, R′ may be CH₃ and R″ may include various substituents. For example, R″ can be: (1) an alkyl of 5 to 12 carbon atoms; (2) a group —O—R′″, wherein R′″ is an alkyl of 5 to 9 carbon atoms; (3) a group —O—R′″, wherein R′″ is an alkyl having a phenyl group at a terminal atom; or (4) a group —(CH₂)ₙ—O-alkyl, wherein n is an integer from 1 to 7 and the alkyl has 1 to 5 carbon atoms. The alkyls of the composition shown in FIG. 1 can have a straight chain or a branched chain. There are many cannabinoid-type compounds that are psychoactive, but this is generally undesired for this purpose and so the composition of the present invention may be non-psychoactive.

FIG. 2 is a structural formula for another example cannabinoid-based pharmaceutical composition for the prevention and/or treatment of GIT conditions, according to embodiments of this invention.

Embodiments of the invention shown in FIGS. 1 and 2 may be formulated for various types of applications. For example, embodiments of the invention may be formulated as external topical application, oral dosage such as powders, granules, tablets, capsules, suspensions, emulsions, syrups, aerosols, suppositories, or in the form of a sterile injectable solution. Acceptable carriers and excipients may include lactose, dextrose, sucrose, sorbitol, mannitol, xylitol, erythritol, maltitol, starches, gum acacia, alginate, gelatin, calcium phosphate, calcium silicate, cellulose, methyl cellulose, microcrystalline cellulose, polyvinylpyrrolidone, water, methyl benzoate, propyl benzoate, talc, magnesium stearate, and mineral oil.

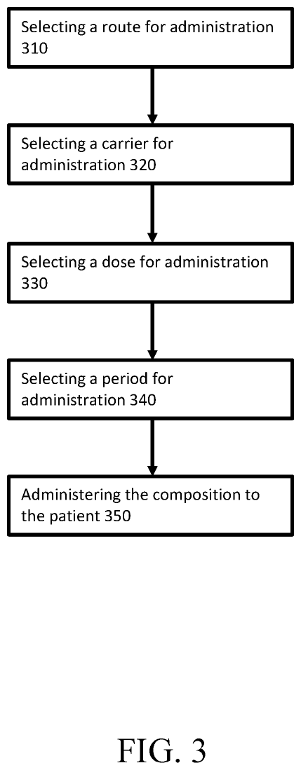

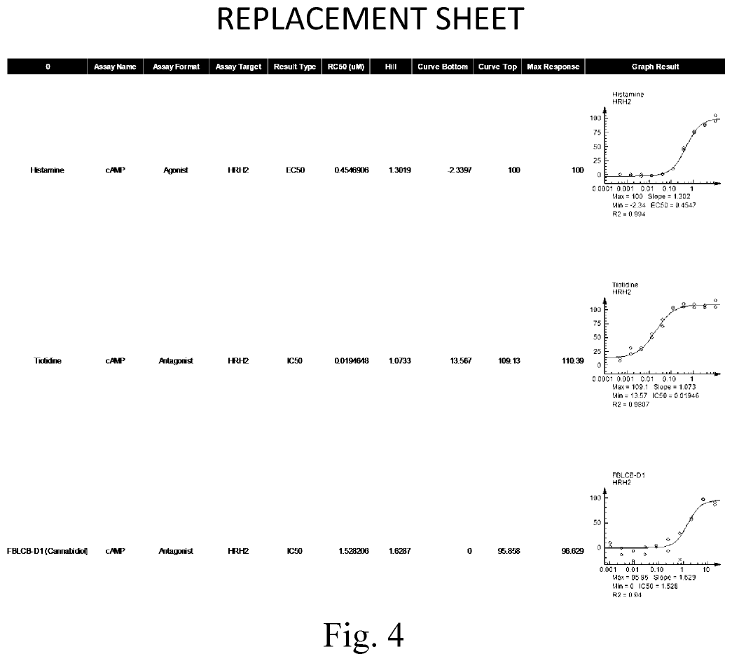

Various therapies may be employed to administer compositions, such as those shown in FIG. 1 and FIG. 2, to patients. FIG. 3 is a flow diagram of an example therapy for the prevention and/or treatment of GIT conditions using the cannabinoid-based pharmaceutical compositions shown in FIGS. 1 and 2, according to embodiments of this invention. When administering the composition, a route of administration may be selected at 310. Routes may include oral, rectal or intravenous, epidural muscle, subcutaneous, intrauterine, or blood vessels in the brain (intracerebroventricular injections). The composition may take on various forms and include various carriers for the composition as discussed further below. At 320 a carrier for the composition may be selected. Selection of the carrier may be based on various factors. For example, the carrier can be selected based on the patient's condition; the patient's body weight, the patient's age, the patient's gender, the patient's health status, the patient's diet, the patient's rate of excretion, an extent of the GIT condition, the route for the administration of the composition, a time of the administration, and/or a duration of the administration.

The cannabinoid derivatives of the formulas shown in FIGS. 1 and 2 or pharmaceutically acceptable salts thereof may be administered in an effective dose. Pharmaceutically-effective doses of the composition may range from 0.1 to 500 milligrams (mg). The dose may be selected at 330 based on various factors. For example, the dose may be selected based on the patient's condition; the patient's body weight, the patient's age, the patient's gender, the patient's health status, the patient's diet, the patient's rate of excretion, the extent of the GIT condition, the carrier of the composition, the route for the administration of the composition, the time of the administration, and/or the duration of the administration.

The compositions can be administered periodically. For example, at 340, a period may be selected, such as between 1-6 times a day. At 350, the composition can be administered to the patient in the form and method selected in accordance with the previous steps. The specific dose level, carrier, and other aspects of the therapy can be changed according to the patient's weight, age, gender, health status, diet, time of administration, method of administration, rate of excretion, severity of disease, and/or other factors. Embodiments of this invention may include additional or fewer steps 310-350 as are suitable for the purposes of this invention. Further, steps 310-350 as well as any additional or fewer steps may be performed in any order that is suitable for the purposes of this invention.

Antagonist activity of the above-described compounds has been verified by use the cyclic adenosine monophosphate (cAMP) secondary messenger pathway using a panel of cell lines stably expressing non-tagged g protein-coupled receptors (GPCRs) that signal through the cAMP. The cAMP assays can monitor the activation of a GPCR via Gi and Gs secondary messenger signalling in a homogenous, non-imaging assay format using, for example, a technology developed by DiscoverX called Enzyme Fragment Complementation (EFC) with β-galactosidase (β-Gal) as the functional reporter.

H2 Blockade Assay

The cannabinoid-based pharmaceutical composition according to FIG. 1 was subjected to an H2 blockade assay. For antagonist determination, cells were pre-incubated with sample followed by agonist challenge at the EC80 concentration. Media was aspirated from cells and replaced with 10 μL 1:1 HBSS/Hepes:cAMP XS+Ab reagent. 5 μL of 4× compound was added to the cells and incubated at 37° C. or room temperature for 30 minutes. 5 μL of 4×EC80 agonist was added to cells and incubated at 37° C. or room temperature for 30 or 60 minutes.

The enzyme is split into two complementary portions, Enzyme Acceptor (EA) and Enzyme Donor (ED). ED is fused to cAMP and in the assay competes with cAMP generated by cells for binding to a cAMP-specific antibody. Active β-Gal is formed by complementation of exogenous EA to any unbound ED-cAMP. The active enzyme can then convert a chemiluminescent substrate, generating an output signal detectable on a standard microplate reader.

Assay Results

FIG. 4 is a table showing an example of antagonist activity of CBD against H2, according to embodiments of this invention. Generally, the inventors have found that CBD is a potent antagonist of H2 in-vitro. CBD inhibited H2 with IC₅₀ of 1.53 μM in a dose-response assay. These findings suggest that CBD can be used therapeutically against GIT conditions, including GERD.

In a preliminary assay, CBD at a single concentration of 10 μM selectively inhibited H2 by 91.5% demonstrating no agonist activity. The molecule also had no agonist or antagonist activity against H1. CBD's close analog THC showed antagonist activity against H1 but none against H2. The single dose activity of CBD and THC activity against H1 and H2 is shown in the table below. Tiotidine was used as a positive control in the concentration dependent assay and had an antagonistic IC₅₀ of 1.07 μM.

| | Compound | | | | | % | % | % |
|---|---|---|---|---|---|---|---|---|
| | Concentration | Target | Assay | Assay | | Response | Response | Response |
| Name | (uM) | Class | Name | Target | Mode | Replicate 1 | Replicate 2 | Average |
| CBD | 10 | GPCR | Calcium | HRH1 | Agonist | 0.3 | 0.2 | 0.3 |
| | | | Flux | | | | | |
| CBD | 10 | GPCR | Calcium | HRH1 | Antagonist | 7.6 | 12.5 | 10.1 |
| | | | Flux | | | | | |
| CBD | 10 | GPCR | cAMP | HRH2 | Agonist | 4.7 | 5.5 | 5.1 |
| CBD | 10 | GPCR | cAMP | HRH2 | Antagonist | | | |
| THC | 10 | GPCR | Calcium | HRH1 | Agonist | −0.5 | −0.9 | −0.7 |
| | | | Flux | | | | | |
| THC | 10 | GPCR | Calcium | HRH1 | Antagonist | 77.0 | 79.3 | 78.2 |
| | | | Flux | | | | | |
| THC | 10 | GPCR | cAMP | HRH2 | Agonist | 4.3 | 2.0 | 3.2 |
| THC | 10 | GPCR | cAMP | HRH2 | Antagonist | −2.0 | 17.3 | 7.7 |

For the purposes of this disclosure, unless expressly stated otherwise: (a) the use of the terms “including,” “having,” and similar terms are deemed to have the same meaning as “comprising” and thus should not be understood as limiting; (b) the term “and/or” means any combination or sub-combination of a set of stated possibilities, for example, “A, B, and/or C,” means any of: “A,” “B,” “C,” “AB,” “AC,” or “ABC;” and (c) headings, numbering, bullets, or other structuring of the text of this disclosure is not to be understood to limit or otherwise affect the meaning of the contents of this disclosure.

The foregoing disclosure, for purpose of explanation, has been described with reference to specific embodiments. The illustrative discussions above are not intended to be exhaustive or to limit embodiments of the invention to the precise forms disclosed. Embodiments of the invention may include combinations or arrangements of multiple embodiments or portions of embodiments of this disclosure in any manner suitable for the purposes of the invention. Many modifications and variations are possible in view of the teachings of this disclosure. The embodiments were chosen and described in order to explain the principles of embodiments of the invention and their practical applications, and to thereby enable others skilled in the art to utilize those embodiments as well as various modifications as may be suited to the particular use contemplated.

We claim:

1. A composition for the treatment of a gastrointestinal tract (GIT) condition, having the following structure or a pharmaceutically acceptable salt thereof: 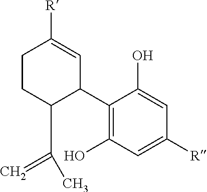
wherein R′ is CH₃, and R″ is selected from a group consisting of:
an alkyl of 5 to 12 carbon atoms,
a group —O—R′″, wherein R′″ is an alkyl of 5 to 9 carbon atoms,
a group —O—R′″, wherein R′″ is an alkyl having a phenyl group at a terminal atom, and
a group —(CH₂)ₙ—O-alkyl, wherein n is an integer from 1 to 7 and the alkyl has 1 to 5 carbon atoms.

2. The composition of claim 1, wherein R″ is the alkyl of 5 to 12 carbon atoms.

3. The composition of claim 1, wherein R″ is the group —O—R′″, wherein R′″ is an alkyl of 5 to 9 carbon atoms.

4. The composition of claim 1, wherein R″ is the group —O—R′″, wherein R′″ is an alkyl having a phenyl group at a terminal atom.

5. The composition of claim 1, wherein R″ is the group —(CH2)n-O-alkyl, wherein n is an integer from 1 to 7 and the alkyl has 1 to 5 carbon atoms.

6. The composition of claim 1, wherein the alkyl forms a branched chain.

7. The composition of claim 1, further comprising a carrier in a form selected from a group consisting of: a powder, a granule, a tablet, a capsule, a suspension, an emulsion, a syrup, an aerosol, a suppository, and an injectable solution.

8. The composition of claim 7, wherein the carrier comprises at least one selected from a group consisting of: lactose, dextrose, sucrose, sorbitol, mannitol, xylitol, erythritol, maltitol, starches, gum acacia, alginate, gelatin, calcium phosphate, calcium silicate, cellulose, methyl cellulose, microcrystalline cellulose, polyvinylpyrrolidone, water, methyl benzoate, propyl benzoate, talc, magnesium stearate, and mineral oil.

9. A composition for the treatment of a GIT condition, having the following structure or a pharmaceutically acceptable salt thereof:

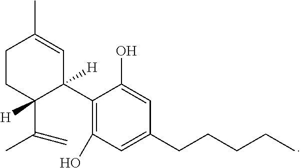 [SMILES: C=C(C)[C@@H]1CCC(C)=C[C@H]1c1c(O)cc(CCCCC)cc1O]

10. The composition of claim 9, further comprising a carrier in a form selected from a group consisting of: a powder, a granule, a tablet, a capsule, a suspension, an emulsion, a syrup, an aerosol, a suppository, and an injectable solution.

11. The composition of claim 10, wherein the carrier comprises at least one selected from a group consisting of: lactose, dextrose, sucrose, sorbitol, mannitol, xylitol, erythritol, maltitol, starches, gum acacia, alginate, gelatin, calcium phosphate, calcium silicate, cellulose, methyl cellulose, microcrystalline cellulose, polyvinylpyrrolidone, water, methyl benzoate, propyl benzoate, talc, magnesium stearate, and mineral oil.

12. A method for treating a GIT condition, comprising administering a therapeutically-effective dose of the composition of claim 1.

13. The method of claim 12, wherein the GIT condition is selected from a group consisting of gastroesophageal reflux disease (GERD), gastritis, acid reflux, and heartburn.

14. The method of claim 13, wherein administering comprises administering via a route selected from a group consisting of: oral, rectal, nasal, intravenous, epidural muscle, subcutaneous, intrauterine, and intracerebroventricular routes.

15. The method of claim 14, further comprising selecting a carrier of the composition for a patient based on at least one factor selected from a group consisting of: the patient's condition; the patient's body weight, the patient's age, the patient's gender, the patient's health status, the patient's diet, the patient's rate of excretion, an extent of the GIT condition, the route for the administration of the composition, a time of the administration, and a duration of the administration.

16. The method of claim 15, further comprising selecting a dose for the patient based on at least one factor selected from a group consisting of: the patient's condition; the patient's body weight, the patient's age, the patient's gender, the patient's health status, the patient's diet, the patient's rate of excretion, the extent of the GIT condition, the carrier of the composition, the route for the administration of the composition, the time of the administration, and the duration of the administration.

17. The method of claim 16, wherein the selected dose is from 0.1 to 500 milligrams (mg) of the composition.

18. The method of claim 17, wherein administering comprises administering periodically from 1 to 6 times a day.

19. The method of claim 18, wherein administering comprises administering the composition to the patient.

20. A method for treating a GIT condition, comprising administering a therapeutically-effective dose of the composition of claim 9.

21. The method of claim 20, wherein the GIT condition is selected from a group consisting of gastroesophageal reflux disease (GERD), gastritis, acid reflux, and heartburn.

22. The method of claim 21, wherein administering comprises administering via a route selected from a group consisting of: topical, oral, rectal, nasal, intravenous, epidural muscle, subcutaneous, intrauterine, and intracerebroventricular routes.

23. The method of claim 22, further comprising selecting a carrier of the composition for a patient based on at least one factor selected from a group consisting of: the patient's condition; the patient's body weight, the patient's age, the patient's gender, the patient's health status, the patient's diet, the patient's rate of excretion, an extent of the GIT condition, the route for the administration of the composition, a time of the administration, and a duration of the administration.

24. The method of claim 23, further comprising selecting a dose for the patient based on at least one factor selected from a group consisting of: the patient's condition; the patient's body weight, the patient's age, the patient's gender, the patient's health status, the patient's diet, the patient's rate of excretion, the extent of the GIT condition, the carrier of the composition, the route for the administration of the composition, the time of the administration, and the duration of the administration.

25. The method of claim 24, wherein the selected dose is from 0.1 to 500 milligrams (mg) of the composition.

26. The method of claim 25, wherein administering comprises administering periodically from 1 to 6 times a day.

27. The method of claim 26, wherein administering comprises administering the composition to the patient.
